(19) United States
(12) Patent Application Publication
(10) Pub. No.: US 2026/0041448 A1
(43) Pub. Date: Feb. 12, 2026

(54) Embolic Protection Device

(71) Applicant: Fortuna Devices, Inc., Birmingham, AL (US)

(72) Inventor: Jan Skowronski, Birmingham, AL (US)

(73) Assignee: Fortuna Devices, Inc., Birmingham, AL (US)

(21) Appl. No.: 19/294,218

(22) Filed: Aug. 7, 2025

Related U.S. Application Data
(60) Provisional application No. 63/680,587, filed on Aug. 7, 2024.

(51) Int. Cl. A61B 17/221; A61B 17/00

(52) U.S. Cl. CPC A61B 17/221; A61B 17/00234; A61B 2017/00238; A61B 2017/00305; A61B 2017/2212

(57) ABSTRACT

The present disclosure includes apparatuses and methods for an embolic protection system. In some embodiments, the system may include a delivery catheter, an embolic protection apparatus, and a retrieval catheter. In some embodiments, an embolic protection apparatus comprises a wire, an expandable loop connected to a porous filter, and a dilator at the distal end of the device.

### CROSS-REFERENCE TO RELATED APPLICATIONS

This application claims the benefit of U.S. patent application Ser. No. 63/680,587, filed Aug. 7, 2024, the entire content of which is hereby incorporated herein by reference.

### TECHNICAL FIELD

This application is directed to embolic protection devices, systems, and methods for providing embolic protection.

### BACKGROUND

Peripheral artery disease (PAD) and coronary artery disease (CAD) affect millions of people in the United States alone. PAD and CAD are silent, dangerous diseases that can have catastrophic consequences when left untreated. CAD is the leading cause of death for in the United States while PAD is the leading cause of amputation in patients.

Coronary artery disease (CAD), Peripheral artery disease (PAD), and Thromboembolic Venous disease (VTE) can be caused by the progressive narrowing of the blood vessels most often caused by atherosclerosis, the collection of plaque or a fatty substance along the inner lining of the artery wall. Over time, this substance hardens and thickens, which may interfere with blood circulation to the arms, legs, stomach and kidneys. This narrowing forms a lesion, completely or partially restricting flow through the artery. Blood circulation to the brain and heart may be reduced, increasing the risk for stroke and heart disease.

The procedures used to treat occlusive vascular diseases, such as angioplasty, atherectomy and stent placement, often cause blood clots to form and/or atheromatous material to dislodge from inside the vessel walls and enter the bloodstream. The dislodged material (e.g., plaque), known as at atheroemboli, may be large enough to occlude downstream vessels, potentially blocking blood flow to tissues. Additionally, the blood clots, known as thromboemboli, may be large enough to block the blood flow downstream. Over the last decade we see fast growth in endovascular treatment of venous conditions including DVT (Deep Venous Thrombosis, PE, (Pulmonary Embolism), and May-Thurner Syndrome. Most these procedures contain Acute Thrombus—all DVT and PE, and some May-Thurner Syndrome cases. In addition, we see increasing number of Iliac Vein stenting in cases of May-Thurner Syndrome treated endovascularly. Some of venous stents re-occlude, and the veins below occluded stents (External Iliac Veins and Femoral Veins) often accumulate large amounts of thrombi. Endovascular treatment of occluded Vein stents is possible in many cases, but is always associated with high risk of embolization and subsequent pulmonary emboli.

There are numerous previously known interventional systems and methods that employ a filter mechanism designed to capture material dislodged from vessel walls during the treatment or diagnosis of vascular disease. Many of the more recent devices employ an expandable filter disposed at the distal end of a guide wire. These filters have various configurations, such as mesh or microporous membranes in the form of sleeves, parachutes or baskets attached to the guide wire or other delivery mechanism by means of struts, wires, ribs or frames. The meshes are frequently made of woven or braided fibers or wires made of stainless steel, nitinol, platinum alloy, polyester, nylon or porous plastics, for example. The microporous membranes are typically made of a polymer material such as polypropylene, polyurethane, polyester, polyethylene terephthalate, polytetrafluoroethylene or combinations thereof.

As the human body has arteries and veins of various sizes, users of embolic protection devices must stock a variety of sizes as know embolic protection devices are indicated only for a small range of vessel sizes. For example, coronary arteries are generally up to 5 mm in diameter and non-aortic peripheral blood vessels are generally up to 10 mm in diameter. Great vessels, such as the vena cava, can have diameters of 20 mm or more. An embolic filter designed to work with a broad range of vessel sizes is described herein.

There remains a need for novel embolic protection devices that are more capable than existing devices, for example being capable of operating under a greater range of conditions and vessel sizes. There remains a need for novel embolic protection devices and delivery systems that are capable of delivering an embolic protection device to a treatment location with reduced interaction with the vessel walls. There remains a need for novel embolic protection devices and delivery systems that allow for the specific placement of the device to be seen, for example on imaging equipment.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### SUMMARY OF THE DISCLOSURE

Described herein are embolic protection devices deployed in a body vessel or cavity for the collection of loosened and/or dislodged debris.

In some embodiments, an embolic protection apparatus comprises a guide wire, a support member, a porous filter and a dilator. In some embodiments, the support member comprises a first leg, a loop and a second leg. The first leg and the second leg are attached to the guide wire, and the loop defines an aperture. The porous filter is attached to the loop and has a conical shape. The dilator is attached to the guide wire distal to the porous filter and comprises a tapered shape.

In some embodiments, a catheter comprises an inner lumen. The support member and porous filter are oriented within the inner lumen and the dilator extends distal to the catheter.

In some embodiments, the dilator comprises a first portion and a second portion. The first portion is oriented within the inner lumen and the second portion extends distal to the catheter. In some embodiments, the dilator seals a distal end of the catheter.

In some embodiments, the catheter comprises a sidewall and an aperture arranged to provide fluid communication through the sidewall. In some embodiments, the aperture is located proximal to the porous filter. In some embodiments, an imaging or contrasting agent is oriented in the aperture.

In some embodiments, the support member includes two legs, connected to the expandable loop, that extend at an angle of approximately 90° from the loop and that are connected to the wire.
[2^{nd} ind]
[3^{rd} ind]

In some embodiments, a method comprises advancing a catheter containing an embolic protection device in an unexpanded orientation to a treatment site in a vessel. The catheter comprises a sidewall and an aperture through the sidewall. In some embodiments, the method comprises imaging the treatment site by injecting a contrasting agent into the catheter, wherein the contrasting agent passes through the aperture. In some embodiments, the method comprises moving the catheter with respect to the embolic protection device to unsheath the embolic protection device and allow expansion thereof.

In some embodiments, the imaging is performed with the embolic protection device in the catheter. In some embodiments, the contrasting agent is oriented in the vessel adjacent to the embolic protection device.

In some embodiments, a method further comprises at least partially resheathing the embolic protection device by moving the catheter with respect to the embolic protection device. In some embodiments, a method further comprises withdrawing the embolic protection device and the catheter from the treatment site.

These and other embodiments which characterize the invention are pointed out with particularity in the claims annexed hereto and forming a part hereof. However, for a better understanding of the invention, its advantages and objectives obtained by its use, reference can be made to the drawings which form a further part hereof and the accompanying descriptive matter, in which there are illustrated and described various embodiments of the invention.

### BRIEF DESCRIPTION OF THE DRAWINGS

FIG. 1 shows an embodiment of an embolic protection device.

FIG. 2 shows another view of the device shown in FIG. 1 in a delivery configuration.

FIG. 3 shows a view similar to FIG. 2 with internal components depicted.

FIGS. 4 and 5 show an embodiment of an embolic protection device during delivery.

FIG. 6 shows an embodiment of an embolic protection device in a deployed configuration in a vessel.

### DETAILED DESCRIPTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the Figures shall refer to like features unless otherwise indicated.

The present disclosure includes methods and apparatuses for devices for providing embolic protection. In some embodiments, an embolic protection device comprises a guide wire with an expandable loop and a filter mounted on the wire near a distal end, and a dilator located distal to the filter. In some embodiments, the expandable loop includes two legs, connected to the expandable loop, that extend at a variable angle from the loop and that are connected to the wire. In some embodiments, the expandable loop comprises an oval or elliptical shape, with the smaller diameter of the loop being perpendicular to the guide wire. In some embodiments, the filter material is provided with a plurality of holes with the holes aligned so as to avoid a perforation or preferential tear line. In one embodiment, the embolic protection device is designed to work in a wide range of lumen diameters.

An example apparatus includes a delivery catheter sized to contain an embolic protection apparatus. In some embodiments, the delivery catheter is used to cross lesions and to be delivered and deployed distal to existing thrombus (distal in respect to blood flow), or the area to be treated with endovascular techniques. In some examples, the distal end of the wire is positioned outside the distal end of the delivery catheter when crossing the lesion. In some examples, the delivery catheter is used to capture the embolic protection device for removal from the body. In some examples, a retrieval catheter is used to capture the embolic protection device for removal from the body.

In the following detailed description of the present disclosure, reference is made to the accompanying drawings that form a part hereof, and in which is shown by way of illustration how one or more embodiments of the disclosure may be practiced. These embodiments are described in sufficient detail to enable those of ordinary skill in the art to practice the embodiments of this disclosure, and it is to be understood that other embodiments may be utilized and that process, electrical, and structural changes may be made without departing from the scope of the present disclosure.

As used herein, designators such as “X”, “Y”, “N”, “M”, etc., particularly with respect to reference numerals in the drawings, indicate that a number of the particular feature so designated can be included. It is also to be understood that the terminology used herein is for the purpose of describing particular embodiments only and is not intended to be limiting. As used herein, the singular forms “a”, “an”, and “the” can include both singular and plural referents, unless the context clearly dictates otherwise. In addition, “a number of”, “at least one”, and “one or more” (e.g., a number of pivot points) can refer to one or more pivot points, whereas a “plurality of” is intended to refer to more than one of such things. Furthermore, the words “can” and “may” are used throughout this application in a permissive sense (i.e., having the potential to, being able to), not in a mandatory sense (i.e., must). The term “include,” and derivations thereof, means “including, but not limited to”. The terms “coupled” and “coupling” mean to be directly or indirectly connected physically or for access to and movement of the movable handle member, as appropriate to the context.

As will be appreciated, elements shown in the various embodiments herein can be added, exchanged, and/or eliminated so as to provide a number of additional embodiments of the present disclosure. In addition, the proportion and/or the relative scale of the elements provided in the Figures are intended to illustrate certain embodiments of the present disclosure and should not be taken in a limiting sense.

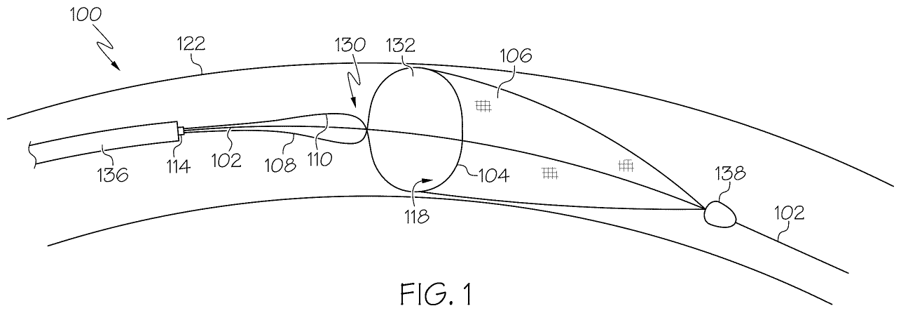

FIG. 1 shows an embodiment of an embolic protection device 100 in a treatment configuration within a vessel 122.

In some embodiments, an embolic protection device 100 comprises a guide wire 102, a support member 130, a filter 106 and a dilator 138. In some embodiments, the support member 130 comprises a loop 104 that defines an aperture 132 leading into a cavity 118 defined by the filter 106. In some embodiments, the filter 106 comprises a porous material constructed an arranged to allow liquids (e.g. blood) to pass through the filter 106 material but to capture solids (e.g. plaque) within the cavity 118. In some embodiments, the filter 106 comprises a conical shape.

In some embodiments, the support member 130 comprises a first leg 108, a loop 104 and a second leg 110. In some embodiments, the support member 130 comprises a single piece of material. In some embodiments, the support member 130 comprises a continuous wire. In some embodiments, the support member 130 is attached at a first end to the guide wire 102 at a junction 114 and extends as the first leg 108 along a length of the guide wire 102, positioned to a first side of the guide wire 102. In some embodiments, the support member 130 then bends and transitions into the loop 104, which comprises an aperture 132 that forms an opening into the cavity 118 of the filter 106. In some embodiments, the support member 130 crosses itself and bends at the transition from the loop 104 into the second leg 110. The second leg 110 can be located to a second side of the guide wire 102, and the second end of the support member 130 can be attached to the guide wire 102 at the junction 114. In some embodiments, a first portion of the support member 130 crosses over a second portion of the support member 130 in a radial direction. In some embodiments, the support member 130 crosses itself at a crossover point, and portions of the support member 130 oriented distal to the crossover point comprise the loop 104, and portions of the support member 130 oriented proximal to the crossover point comprise the first leg 108 and second leg 110. In some embodiments, the crossover point moves along the length of the support member 130 as the loop 104 changes in size.

In some embodiments, the first leg 108 is positioned on a first side of a bisecting longitudinal plane and the second leg 110 is positioned on the second side. In some embodiments, the support member 130 crosses the bisecting longitudinal plane at the transition from the first leg 108 into the loop 104. Thus, in some embodiments, the first leg 108 is attached to a portion of the loop 104 oriented on the second side of the bisecting longitudinal plane. In some embodiments, the loop 104 crosses the bisecting longitudinal plane at a location across the aperture 132 from the legs 108, 110. In some embodiments, the support member 130 again crosses the bisecting longitudinal plane at the transition from the loop 104 into the second leg 110. Thus, in some embodiments, the second leg 110 is attached to a portion of the loop 104 oriented on the first side of the bisecting longitudinal plane. Embodiments where the legs 108, 110 cross one another when transitioning to the loop 104 allow for a greater reduction in size of the device 100, and allow the device 100 to be used in a greater range of vessel sizes.

A junction 114 can comprise any suitable type of attachment, including a weld, glue, or other bonding agent or can comprise a sleeve heat sealed, shrunk, or bonded over legs 108 and 110 and to the guide wire 102, or any other suitable method.

In some embodiments, the aperture 132 is oriented at an angle to the legs 108, 110, for example being arranged at an angle of 70° to 90° to the legs 108, 110. In some embodiments, the expandable frame 104 comprises a material that has been heat set to a predetermined shape, for example an expanded shape.

In some embodiments, the shape of the support member 130 allows the embolic protection device 100 to be used in vessels having a range of sizes as the specific area of the aperture 132 is variable. For example, in some embodiments, the expandable frame 104 is manufactured to have a predetermined shape, and it will assume the predetermined shape when not being constrained by external forces. When the embolic protection device 100 is used in vessels that are smaller than the size of the predetermined shape, the aperture 132 will expand from its compressed delivery orientation to the proper size necessary and match the size of the vessel. A predetermined shape can be imparted to the expandable frame 104 using any suitable method. In some embodiments, the expandable frame 104 is heat set to retain the predetermined shape. In some embodiments, the expandable frame comprises a shape memory material such as nickel titanium, and is programmed to return to the predetermined shape.

In some embodiments, the support member 130 is attached to the guide wire 102 only at the junction 114. Thus, in some embodiments, the legs 108, 110 are only attached to the guide wire 102 at their proximal ends and the loop 104 is not specifically attached to the guide wire 102. This configuration allows for greater flexibility for the loop 104 to adjust in size in response to smaller vessels, as the loop 104 is able to move away from the guide wire 102. Also, the legs 108, 110 are able to pivot at the junction 114, allowing the distal ends of the legs 108, 110 to move away from the guide wire 102 with the loop 104, and also allowing the distal ends of the legs 108, 110 to spread laterally as the loop 104 is compressed in size.

In some embodiments, a first end of the filter 106 material is attached to the loop 104 around its perimeter and the aperture 132 comprises an entrance into the cavity 118 of the filter 106. In some embodiments, the guide wire 102 passes through the interior of the aperture 132 and extends inside of the filter 106 cavity. In some embodiments, a distal end of the filter 106 is attached to the guide wire 102.

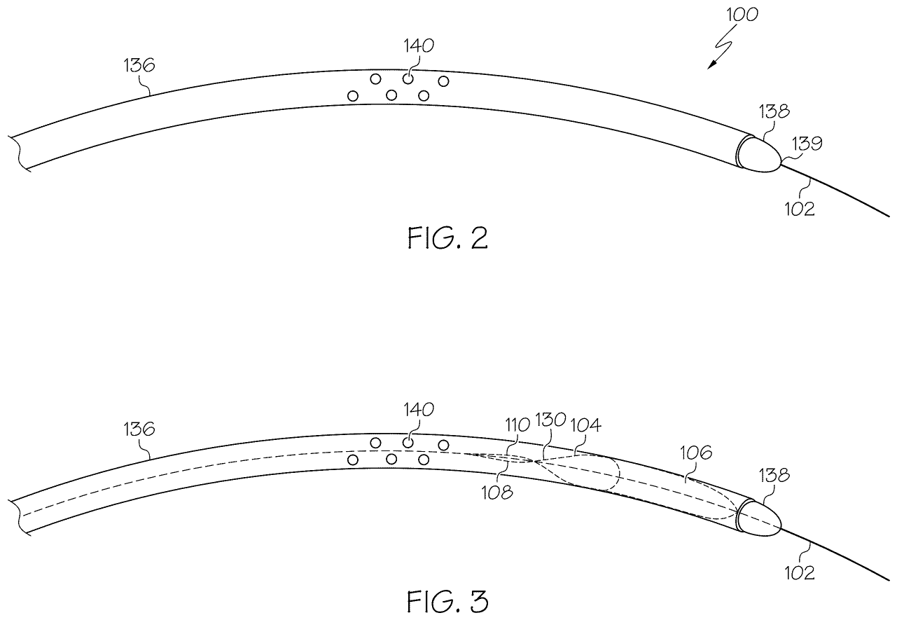

FIG. 2 shows the device of FIG. 1 in a delivery configuration. FIG. 3 shows a view similar to FIG. 2, but illustrates components contained within a catheter 136.

In some embodiments, an embolic protection device 100 comprises a catheter 136. In some embodiments, the catheter 136 comprises a delivery catheter arranged to deliver the embolic protection device 100 in a collapsed configuration through a vessel. In some embodiments, the catheter 136 comprises a tube defining an internal lumen, and portions of the guide wire 102, support member 130, filter 106 and dilator 138 can be oriented within the lumen. In some embodiments, the catheter 136 constrains the support member 130 and retains it in an unexpanded configuration. In some embodiments, the embolic protection device 100 can be advanced out of the distal end of the catheter 136 by moving the guide wire 102 with respect to the catheter 136. In some embodiments, when the support member 130 exits the catheter 136, it will attempt to return to its predetermined shape, and the aperture 132 will automatically assume the size of the vessel 122.

In some embodiments, a dilator 138 comprises a tip 139 of the device 100 that gradually increases in size. In some embodiments, the dilator is tapered. In some embodiments, a forward end (e.g. distal end) of the dilator 138 is approximately the size of the guide wire 102, and a diameter of the dilator 138 increases along the length of the dilator 138. In some embodiments, the dilator 138 increases in size to a largest diameter portion diameter. In some embodiments, the largest diameter portion is located at the rear end (e.g. proximal end) of the dilator 138. In some embodiments, the dilator 138 reaches its largest diameter portion and comprises a reverse taper, wherein the rear end of the dilator 138 comprises a size that is less than the largest diameter portion. In some embodiments, a dilator 138 comprises a shape that is conical, ovular, elliptical, convex, or any other suitable shape that increases in size along the length of the dilator 138, for example as the dilator 138 is traversed from the tip 139 toward the catheter 136.

In some embodiments, the dilator 138 is sized to fit within the internal lumen of the catheter 136 and the largest portion diameter of the dilator 138 is approximately equal to, or slightly less than, an inner diameter of the catheter 136. In some embodiments, a cross-sectional area of the dilator 138 is sized to occlude, or nearly occlude, the catheter 136. For example, in some embodiments, a cross-sectional area of the dilator 138 is at least 90% of the cross-sectional area of the inner lumen of the catheter 136. In some embodiments, an outer diameter of the dilator 138 contacts an inner sidewall of the catheter 136. In some embodiments, a dilator 138 can be larger than the catheter 136 and can be designed to abut the distal end of the catheter 138.

In some embodiments, a first portion of the dilator 138 is oriented within the inner lumen of the catheter 136 and a second portion of the dilator 138 extends distal to an end of the catheter 136. In some embodiments, the first portion of the dilator 138 comprises the maximum diameter of the dilator 138. In some embodiments, the second portion of the dilator 138 comprises a taper.

In some embodiments, a dilator 138 is located directly adjacent to a distal end of the filter 106. In some embodiments, a dilator 138 can contact a distal end of the filter 106.

In some embodiments, a dilator 138 is arranged to enhance deliverability of the embolic protection device 100 to a treatment location. For example, a dilator 138 can help the embolic protection device 100 to cross lesions or occlusions in a vessel, and prevent a distal end of the catheter 136 from disrupting thrombus or injuring the vessel wall.

In some embodiments, a dilator 138 is arranged to reduce contact between a vessel wall and the catheter 136, to prevent injury to branch vessels and to allow for atraumatic delivery of the embolic protection device 100.

In some embodiments, a catheter 136 comprises at least one aperture 140. In some embodiments, a catheter 136 comprises a plurality of apertures 140. In some embodiments, the aperture(s) 140 allow fluid communication through the sidewall of the catheter 136, for example, fluid communication between the inner lumen of the catheter 136 and the environment surrounding the catheter 136. In some embodiments, an aperture 140 is oriented in a radial direction of the catheter 136, for example wherein a central axis of the aperture 140 extends radial to the catheter 136. In some embodiments, a contrasting agent can be provided in the inner lumen of the catheter 136, for example by using a Tuohy-Borst Adapter. The contrasting agent can be ejected from the catheter 136 through the aperture(s) 140, allowing the contrasting agent to be viewed as an angiographic image, such as a venogram.

In some embodiments, the support member 130 and filter 106 are contained within the catheter 136, and an aperture 140 is located proximal to the support member 130. In some embodiments, an aperture 140 is located proximal to the filter 106. In some embodiments, an aperture 140 overlaps with the support member 130 along a length of the device 100. By placing the aperture(s) 140 proximal to the support member 130, and/or overlapping with the support member 130, a contrasting agent allows for visualization of an approximate location of the support member 130 in reference to body anatomy, like Inferior Vena Cava or Iliac Vein. This allows for more precise visualization of the positioning of the device 100 than if contrasting agent were ejected from the distal end of the catheter 136. When contrast is injected into the catheter 136 it will exit through apertures 140, as the distal end of the lumen of catheter 130 is occluded by dilator 138. This allows for imaging without removing the embolic protection device 100 from the catheter, and avoids repeated introduction and removal of the embolic protection device 100 into the catheter 136 in order to obtain angiographic image

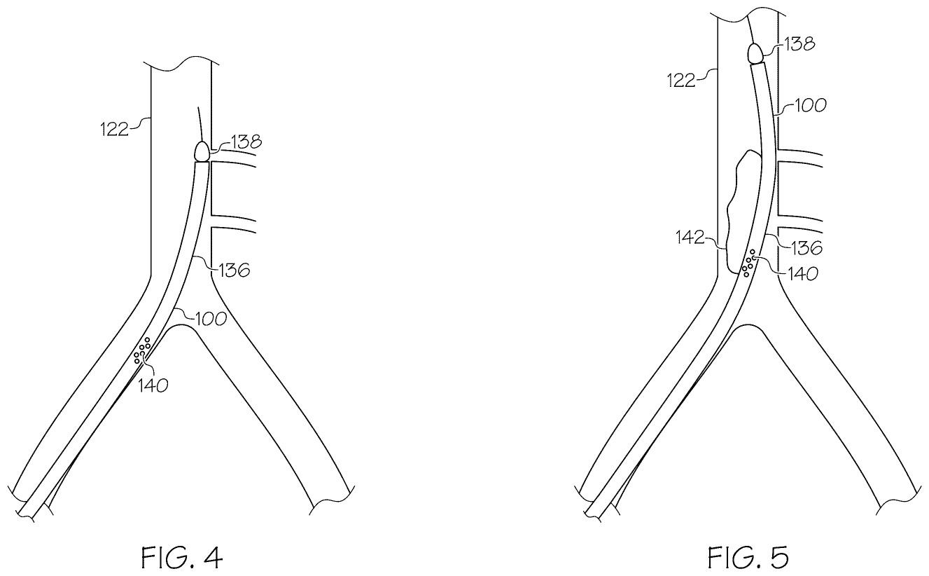

FIG. 4 shows an embodiment of an embolic protection device 100 during delivery to a treatment location in a vessel 122. As the embolic protection device 100 is advanced through a tortuous anatomy, the dilator 138 can contact vessel walls and branching vessel structures. Desirably, the shape of the dilator 138 allows the embolic protection device 100 to be advanced with reduced amounts of stress applied to vessel walls. For example, in some embodiments, a tapered shape of the dilator 138 encourages advancement of the embolic protection device 100 and prevents a distal end of the catheter 136 from contacting the vessel 122.

FIG. 5 shows an example of an imaging fluid 142 being used, for example to visualize the embolic protection device 100 and vessel 122 anatomy on an angiographic image. In some embodiments, the embolic protection device 100 can be advanced to an approximate treatment location. An imaging fluid 142 can be delivered to the treatment location using an internal lumen of the catheter 136. In some embodiments, the imaging fluid 142 passes through aperture(s) 140 formed through the sidewall of the catheter 136. In some embodiments, downstream bloodflow within the vessel 122 will carry the imaging fluid 142 in a distal direction. By having the aperture(s) 140 positioned proximal to the support member 130 and filter 106, the imaging fluid 142 will indicate an approximate location of the support member 130, filter 106 and vessel 122 anatomy.

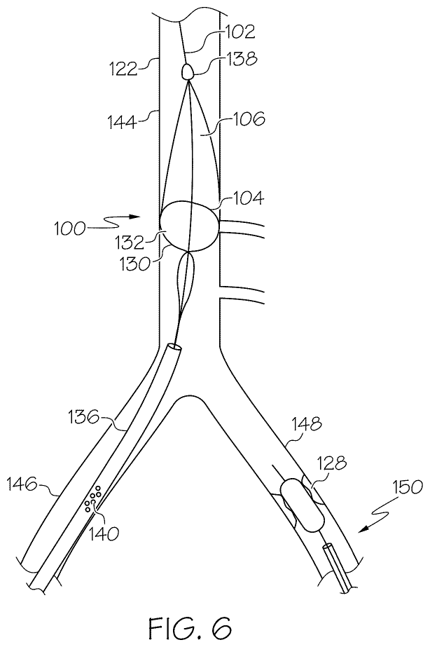

FIG. 6 shows an embodiment of an embolic protection device 100 deployed in a vessel 122, such as an inferior vena cava 144. In some embodiments, an embolic protection device 100 is constructed and arranged to be delivered through a first branch vessel 146, such as a first iliac vein, to a treatment location in the inferior vena cava 144. In some embodiments, an imaging fluid 142 can be used to visualize a location of the embolic protection device 100 and vessel 122 anatomy. In some embodiments, the support member 130 and filter 106 can be unsheathed by retracting the catheter 136, allowing the expandable loop 104 to deploy. In some embodiments, the loop 104 of the support member 130 will expand to match a diameter of the inferior vena cava 144 or iliac vein 146. The embolic protection device 100 is then arranged to capture material, such as plaque, thrombus, which would otherwise flow through the inferior vena cava 144. In some embodiments, a medical procedure 150, such as an angioplasty, can be performed in a second branch vessel 146 or 148, such as an ipsilateral or contralateral iliac vessel. Thus, in some embodiments, an embolic protection device 100 can be delivered via a first iliac vessel 146 and a medical procedure 150 can be performed in an iliac vessel 148 or Inferior Vena Cava. In some embodiments, the catheter 136 of the embolic protection device 100 remains in the iliac vessel 146 as the medical procedure 150 is performed in the second iliac vessel 148. In some embodiments, the embolic protection device 100 is arranged to capture any plaque or thrombus 128 that becomes dislodged during the medical procedure 150.

In various embodiments, the embolic protection device 100 can have an aperture 132 having any suitable expanded size. In some embodiments, an expanded diameter of the aperture 132 is at least 25 mm.

In various embodiments, a size of the catheter 136 can be selected based upon size of the support member 130 and filter 106. In some embodiments, a catheter 136 comprises a size of 8-14 French. In some embodiments, a catheter comprises a size of 10-12 French. In some embodiments, a dilator 138 is sized to fit within an inner lumen of the catheter 136. In some embodiments, a diameter of the dilator 138 is approximately 2 French less than the size of the catheter 136.

In some embodiments, a filter 106 comprises thermoplastic polymers or thermoplastic elastomers. In some embodiments, a filter 106 comprises thermoplastic polyurethanes or thermoplastic polyurethane elastomers. In some embodiments, a filter 106 comprises polyurethane. In some embodiments, a filter 106 comprises thermoplastic polyurethanes including aromatic thermoplastic polyurethanes. In some embodiments, a filter 106 is made from a flat piece of polymer that is rolled and sealed in its conical shape. In some embodiments, a filter 106 is formed in a conical shape. In some embodiments, the thickness of filter 106 is 0.02 to 0.1 mm or from 0.025 to 0.05 mm. In one embodiment, the thickness of filter 106 is between 0.03 mm and 0.04 mm. In some embodiments, holes 112 in the filter 106 can have a diameter of 0.1 to 0.2 mm, or from 0.14 to 0.16 mm. In one embodiment, the diameter of holes 112 is 0.15 mm. In some embodiments, the hole center to hole center spacing of holes 112 is 0.2 to 0.5 mm or from 0.3 to 0.4 mm. In one embodiment, the hole center to hole center spacing of holes 112 is 0.35 mm.

In some embodiments, the embolic protection device 100 is designed to work in a large range of lumen diameters. In one example, device 100 will work in lumens ranging from 12 to 25 mm. When positioned in lumens, legs 110 and 108 can be approximately or substantially parallel to wire 102. When positioned in smaller diameter lumens, legs 108 and 110 extend at an angle from wire 102. The proximal ends of legs 108 and 110 are attached to wire 102 at 114. When positioned in a smaller diameter lumen, each of legs 108 and 110 can extend from wire 102 at an angle of 5° to 25°.

In some embodiments, an embolic protection device 100 is capable of assuming several different deployed orientations, for example in response to external forces applied by various sized vessels 122. In some embodiments, an embolic protection device 100 comprises a first orientation and a second orientation. In some embodiments, a cross-sectional area of the aperture 132 defined by the loop 104 is larger in the first orientation and smaller in the second orientation. In some embodiments, an angle between the legs 108, 110, for example measured from the junction 114, is greater in the second orientation than in the first orientation. In some embodiments, a spacing between the proximal ends of the legs 108, 110 is greater in the second orientation than in the first orientation. In some embodiments, an angle between a portion of the guide wire 102 located between the legs 108, 110 and the cross-sectional face of the aperture 132 comprises an oblique angle. In some embodiments, the angle between the guide wire 102 and the cross-sectional face of the aperture 132 is greater in the second orientation than in the first orientation. In some embodiments, the loop 104 and legs 108, 110 collectively pivot away from the guide wire 102 at the junction 114 as the support member 130 deforms in response to external forces. Thus, in some embodiments, the distal ends of the legs 108, 110, and a portion of the loop 104, are spaced farther away from the guide wire 102 in the second orientation than in the first orientation. In some embodiments, the filter 106 generally comprises a single layer of material at its connection to the loop 104 in the first orientation. In some embodiments, a portion of the filter 106 can fold over itself and form an overlap 134 in the second orientation. In some embodiments, the overlap 134 is located adjacent to the loop 104. In some embodiments, an overlap 134 is located opposite from the legs 108, 110.

In some embodiments, after an intervention procedure is complete, the catheter 136 that was used for delivery of the embolic protection device 100 can be used to remove the embolic protection device 100. For example, the embolic protection device 100 can be retracted into the catheter 136. Desirably, at least a portion of the support member 130 can be retracted within the internal lumen of the catheter 136, such that the size of the aperture 132 is reduced. In some embodiments, the entire support member 130 is retracted into the catheter 136. The catheter 136 and embolic protection device 100 can then be removed from the vessel 122.

In some embodiments, a second catheter (not illustrated) can be used to remove the embolic protection device 100. In some embodiments, a retrieval catheter having a larger size than the delivery catheter (e.g. 136) can be used to retrieve the embolic protection device 100. In some embodiments, a retrieval catheter can be advanced over the delivery catheter. In some embodiments, the deliver catheter can be removed before the retrieval catheter is advanced to the treatment site.

U.S. patent application Ser. No. 17/556,967, published as US2022/0192689, is hereby incorporated herein by reference in its entirety. U.S. patent application Ser. No. 18/213,178, published as US2023/0414337, is hereby incorporated herein by reference in its entirety.

The device described herein may be used for specific clinical indications. In some embodiments, the device can serve as peripheral embolic protection device. In some embodiments, the device can serve as a temporary IVC filter. Many endovascular procedures create unacceptable risk for peripheral embolizations, and many peripheral procedures are performed in presence of existing thrombus. The device will protect the patient from the risk of atheroemboli, and thromboemboli. Deployment of embolic protection basket distal to the lesion/thrombus will mitigate the risk of embolic complications during endovascular procedures. Its design and size can be tailored to peripheral veins, including Inferior Vena Cava, Superior Vena Cava, Iliac Veins, Innominate Vein, Subclavian Veins.

The apparatuses of this disclosure are useful in a number of clinical situations. The apparatus described herein is useful in the venous system and can be used to treat lesions in the iliac, femoral, popliteal, brachial, subclavian, axillary, innominate veins, and in the Inferior Vena Cava as well as Superior Vena Cava. Depending on the clinical requirements, either a radial, brachial, subclavian, pedal, proximal tibial, or femoral access can be used.

While many of the examples herein show and describe the devices and methods being used and performed in the vascular system, the devices and methods have applicability to non-vascular lumens.

The delivery catheter and embolic protection apparatus will be constructed from materials that are known in the art. The delivery and retrieval catheters may have a multilayer or single layer construction. In a multilayer construction, the catheter could have a polymer inside layer, surrounded by a support structure such as a metal braid which in turn is surrounded by an outer polymer layer. Either catheter could have a flexibility that is consistent over the length of the catheter or could have increased flexibility at the distal end. Alternatively, the catheters could be made from a single or multi-stream extrusion, with or without an internal support structure. When one or both of the delivery and retrieval catheters have one or more marker bands, the marker bands can be formed of any radiopaque material and be in the form of a ring attached to either the internal or external surface, embedded in the internal or external surface so that they are flush with the surface, embedded within the wall structure of the catheter, or be a radiopaque agent mixed with the plastic of the catheter. One or both of the delivery and retrieval catheters can have a distal tip that is softer and/or more flexible that the body of the catheter. The embolic protection filter wire can be constructed of superelastic materials, nitinol, stainless steel, cobalt-chromium-nickel-molybdenum-iron alloy, or cobalt-chrome alloy, or a combination thereof. In embodiments where the basket and/or distal section(s) of the wire have a lesser diameter than the proximal section, the smaller diameter can be achieved by grinding or milling of the wire or by attaching a smaller diameter wire to the distal end of a larger diameter wire. The expandable loop can be constructed from superelastic materials, nitinol, stainless steel, cobalt-chromium-nickel-molybdenum-iron alloy, or cobalt-chrome alloy, or a combination thereof.

The porous filter 106 can be fabricated from a variety of different materials, such as, but not limited to, a woven or braided plastic or metallic mesh, a perforated polymer film, a shape memory material or mesh, combinations thereof, or other material that can be capable of capturing material within flowing blood, while allowing the blood to flow through the pores of the material. In some embodiments the porous filter comprises expanded polytetrafluoroethylene (ePTFE), polyurethane, polyolefin elastomers, polyamides, nylons, polyethers, polyamide block ethers (PEBAX), polyesters, and/or co-polyesters. In some embodiments the filter material has a thickness of 0.001 inches (25 microns) and the material has an 85 A Shore A Hardness. In some embodiments the filter material has a thickness of 0.0017 inches with an 80 Shore A Hardness. In some embodiments the porous filter can be woven or braided into a mesh and can be made from polyester, polyamide, polyurethane, nitinol, or stainless-steel filaments. The porous filter can have a variety of differently sized pores ranging from about 50 microns to about 200 microns, from about 60 microns to about 180 microns, or from about 75 microns to about 150 microns. For some applications, the pores can be sized up to 250 microns. The pores can have a variety of different configurations and can be circular, oval, polygonal, combinations thereof and the porous filter can include pores that are differently sized and configured. In practice, the pore size can vary as needed, so long as the pores are sized so that the pores do not compromise blood flow through the filter and collect emboli that can adversely affect downstream vessels. The porous filter can be coated with a hydrophilic coating, a heparinized coating, PTFE, silicone, combinations thereof, or other coatings. In some embodiments, the porous filter can be attached to the expandable loop by dip coating or by being wrapped around the loop and then sealed with heat or through an adhesive.

In some embodiments, a method comprises advancing a catheter 136 containing an embolic protection device 100 in an unexpanded orientation to a treatment site in a vessel 122. In some embodiments, the catheter 136 comprises a sidewall and an aperture 140 through the sidewall. The aperture 140 desirably provides fluid communication between an internal lumen of the catheter 136 and the area external to the catheter 136, such as an internal lumen of the vessel 122. In some embodiments, the method comprises imaging the treatment site, for example by injecting a contrasting agent 142 into the catheter, wherein the contrasting agent 142 passes through the aperture 140 and into the vessel 122. In some embodiments, imaging is performed before expansion of the embolic protection device 100. In some embodiments, the imaging is performed with the embolic protection device 100 in the unexpanded orientation positioned within the catheter 136. In some embodiments, the contrasting agent 142 is oriented in the vessel 122 adjacent to the catheter 136 and adjacent to the unexpanded embolic protection device 100. In some embodiments, the contrasting agent 142 is positioned in the aperture 140 adjacent to the unexpanded embolic protection device 100. In some embodiments, the method comprises unsheathing the embolic protection device 100, wherein the embolic protection device 100 expands, for example by moving the catheter 136 (e.g. retracting) with respect to the embolic protection device 100. In some embodiments, a medical procedure is performed when the embolic protection device 100 is expanded within the vessel 122. In some embodiments, the embolic protection device 100 is positioned to capture material (e.g. thrombus) generated or dislodged by the medical procedure. In some embodiments, the method comprises at least partially resheathing the embolic protection device 100, for example by moving the catheter 136 (e.g. advancing) with respect to the embolic protection device 100. In some embodiments, the method comprises withdrawing the embolic protection device 100 and the catheter 136 from the treatment site. In some embodiments, the catheter 136 used to deliver the embolic protection device 100 is used to resheath and withdraw the embolic protection device 100.

In some embodiments, a method for protecting a vessel comprises providing an embolic protection device comprising a wire with a support member positioned near a distal end of the wire. The support member comprises an expandable loop with two legs, which are connected to the wire at a connection. A porous filter is attached at one end to the loop and a dilator is positioned at the end of the porous filter. In some embodiments, the two legs extend along the wire between the support member and the connection. In some embodiments, the embolic protection device is positioned within a delivery catheter with the dilator positioned at the distal end of the catheter. The delivery catheter and the embolic protection device are advanced through a lumen to a position downstream from a treatment area. During advancement, the dilator can contact a vessel wall and reduce interactive forces between the catheter and the vessel. The delivery catheter is withdrawn to allow the expandable loop to expand into contact with the interior of the lumen.

Although specific embodiments have been illustrated and described herein, those of ordinary skill in the art will appreciate that an arrangement calculated to achieve the same results can be substituted for the specific embodiments shown. For example, where the disclosure my show a system or a method with one example of a distal protection device, any distal protection device can be used including those disclosed herein. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and processes are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. An embolic protection apparatus comprising:
a guide wire;
a support member comprising a first leg, a loop and a second leg, the first leg attached to the guide wire, the loop defining an aperture, the second leg attached to the guide wire;
a porous filter attached to the loop, the porous filter comprising a conical shape; and
a dilator attached to the guide wire distal to the porous filter, the dilator comprising a tapered shape.

2. The embolic protection apparatus of claim 1, comprising a catheter comprising an inner lumen, the support member and porous filter oriented within the inner lumen, the dilator extending distal to the catheter.

3. The embolic protection apparatus of claim 2, the catheter comprising a sidewall and an aperture arranged to provide fluid communication through the sidewall.

4. The embolic protection apparatus of claim 3, the aperture located proximal to the porous filter.

5. The embolic protection apparatus of claim 3, comprising a contrasting agent oriented in the aperture.

6. The embolic protection apparatus of claim 3, wherein the dilator seals a distal end of the catheter.

7. The embolic protection apparatus of claim 2, the dilator comprising a first portion and a second portion, the first portion oriented within the inner lumen, the second portion extending distal to the catheter.

8. The embolic protection apparatus of claim 1, the dilator comprising a convex shape.

9. An embolic protection apparatus comprising:
a guide wire;
a support member comprising a first leg, a loop and a second leg, the first leg attached to the guide wire, the second leg attached to the guide wire, the support member crossing itself in a radial direction at a crossover point, the first leg and the second leg oriented proximal to the crossover point, the expandable loop oriented distal to the crossover point, the crossover point moving along a length of the support member as the expandable loop changes in size;
a porous filter attached to the loop and extending distal to the loop;
a catheter, the support member and porous filter oriented within the catheter; and
a dilator attached to the guide wire, the dilator positioned distal to the porous filter, at least a portion of the dilator located distal to the catheter.

10. The embolic protection apparatus of claim 9, the dilator comprising a convex shape.

11. The embolic protection apparatus of claim 9, the catheter comprising a sidewall and an aperture arranged to provide fluid communication through the sidewall.

12. The embolic protection apparatus of claim 11, the aperture located proximal to the porous filter.

13. The embolic protection apparatus of claim 11, comprising a contrasting agent oriented in the aperture.

14. The embolic protection apparatus of claim 11, wherein the dilator seals a distal end of the catheter.

15. The embolic protection apparatus of claim 9, the dilator comprising a first portion and a second portion, the first portion oriented within the catheter, the second portion extending distal to the catheter.
